(19) United States
(12) Patent Application Publication
(10) Pub. No.: US 2007/0074730 A1
(43) Pub. Date: Apr. 5, 2007

(54) CONDUCTIVE KERATOPLASTY PROBE GUIDE DEVICE AND METHODS THEREOF

(76) Inventors: Padma NANDURI, LaJolla, CA (US); T. Aaron DYER, LaJolla, CA (US)

Correspondence Address: GODFREY & KAHN S.C., 780 NORTH WATER STREET, MILWAUKEE, WI, 53202, US

(21) Appl. No.: 11/163,043

(22) Filed: Oct. 3, 2005

(51) Int. Cl. A61B 19/00; A61B 18/18

(52) U.S. Cl. 128/897; 606/41

(57) ABSTRACT

The present invention provides a biocompatible Conductive Keratoplasty probe guide device having an arcuate member and at least one orifice capable of allowing the probe to be inserted through the orifice. Also, the present invention teaches methods related to guiding a CK probe through this probe guide device.

### FIELD OF INVENTION

The present invention generally relates to Conductive Keratoplasty, and specifically to probe guide devices and methods useful in improving the results of the Conductive Keratoplasty.

### BACKGROUND

Conductive Keratoplasty®, or CK (Conductive Keratoplasty, CK and Keratoplast are registered trademarks of Refractec, Inc, Irvine, Calif.), is a thermal keratoplasty technology that uses low energy radio frequency (RF) current to shrink collagen, and is included in the term “RF thermal keratoplasty (RFTK)”. The low energy radiofrequency (RF) electric current is delivered directly into the corneal stroma through a hand piece and Keratoplast™ Tip, to produce refractive changes in the cornea. As a result of conducting a controlled amount of RF energy into the corneal stroma, the desired collagen shrinkage temperature is achieved. The peripheral application of this treatment in a predetermined pattern creates a band of tightening and results in steepening of the central cornea. This steepening in turn results in the desired refractive effect, for example, in the treatment of hyperopia and presbyopia.

In order to improve the accuracy and standardization of a CK probe, an inked marker is used, however during the treatment these ink marks may be obliterated resulting in variability and over corrections and increased astigmatism. In order to reduce problems associated with inking, an injection molded plastic marker, for example ACCUMARCK™, may be used. (ACCUMARCK is a trademark of International Science and Technology, LP Diamatrix Ltd, Inc, Texas) This plastic marker that does not require inking and can be placed on the wet cornea to produce 32 marks that are long lasting and readily visible. While this can aid the appropriate and efficient placement of the CK probe, it does not solve the problem of optimizing probe angle or depth for RF application.

In practice, a surgeon typically applies a pen-shaped probe at 6, 7, and 8 mm radius of the cornea relative to the center of the cornea as defined by the center of the pupil. Generally, the probe is applied freehand and any tilting of the probe or movement of the eye by the patient can alter the angle of the probe. This alteration has unintended consequences of inducing astigmatism, producing ghosting of vision or doubling of images and a unpredictable refractive outcome.

Accordingly, the need exists for a CK guide device that would minimize variability of outcomes by optimizing the angle and depth of application of RF energy, so as to reduce induced astigmatism and associated problems, such as visual deficits and negative visual outcomes.

### SUMMARY OF THE INVENTION

The present invention generally provides a biocompatible ophthalmic probe guide device having an arcuate member and at least one orifice disposed to admit and align a tip of an ophthalmic probe. In several preferred embodiments, the ophthalmic probe guide device is a conductive keratoplasty probe guide device having an arcuate member and at least one orifice capable of allowing a conductive keratoplasty probe to be inserted through the orifice. Also, the present invention teaches methods related to guiding a CK probe through this probe guide device. In other embodiments, the probe guide device is useful for guiding an ophthalmic surgical instrument for any corneal or limbal incision or an ophthalmic probe for procedures such as cataract surgery, astigmatic keratotomy, radial keratotomy, thermal keratoplasty, lamellar keratoplasty, scleral ports, or sclerectomy In such probe guide devices the arcuate member is configured to contact the region of cornea or sclera to be penetrated and the orifice is configured to admit and align the corresponding ophthalmic probe or instrument.

In one preferred embodiment, the biocompatible ophthalmic conductive keratoplasty (CK) probe guide device comprises an arcuate member having a top surface and a bottom surface. In such an embodiment, the arcuate member has at least one orifice between the top and the bottom surfaces and at least one alignment index, such as a cross hair, on the top or the bottom surface.

Preferably, the probe guide device has at least 24 orifices arranged in a symmetrical pattern. The arrangement of the orifices may be as follows: the first 8 orifices are located at about 6 mm distance from the center of the cross hair, the second 8 orifices are located at about 7 mm distance from the center of the cross hair and the last 8 orifices are located at about 8 mm distance from the center of the cross hair. This arrangement forms 8 radial arrays of 3 orifices each. Further, the 8 radial arrays are preferably substantially equiangularly positioned at 45° to each other.

In another preferred embodiment, the probe guide device has at least 16 orifices arranged in a symmetrical pattern such that the first 8 orifices are located at about 6.5 mm distance from the center of the cross hair and the second 8 orifices are located at about 7.5 mm distance from the center of the cross hair. Preferably, this forms 8 radial arrays of 2 orifices each. Also preferably, these 8 radial arrays are substantially equiangularly positioned at 45° to each other.

Also, in some preferred embodiments of such a probe guide device, the orifice is substantially cylindrical in shape. In one preferred embodiment, the orifice 12 has a diameter of 400 to 600 microns and a depth of 400 to 600 microns. In another preferred embodiment, the orifice has a diameter of about 90 to about 100 μm and depth of about 450 to about 500 μm. In certain preferred embodiments, the tip of the CK probe is inserted into an orifice of the device so that the tip of the probe indents the cornea to the extent that the base of the probe tip is flush with the corneal surface. The RF energy is applied while the base of the probe tip is flush with and compressing the cornea. In such embodiments, the orifice of the CK probe guide device has an internal diameter suitable to accommodate the outside diameter of the base of the probe tip, typically 400-600 μm, and preferably 450-550 μm and a depth about the length to the base of the probe tip, typically 400-600 μm, and preferably 450-550 μm.

In other preferred embodiments, “light touch CK” (sometimes called “neutral touch CK”) is performed in which the probe tip is firmly placed into the cornea at each spot using only adequate pressure to indent to the point that striae from the corneal compression extends to the pupil and such that the ring light reflection is displaced away from the probe. In such embodiments, the orifice of the CK probe guide device has an internal diameter suitable to accommodate the outside diameter of the probe tip, typically 80-120 μm, preferably 90-110 μm, more preferably 95-105 μm and a depth that can be less than the distance to the junction of the tip and the base of the probe tip, typically less than 400-600 μm, and preferably less than 400-500 μm.

Typically the “standard CK” method would require the orifice 12 dimensions to be 400 to 600 μm in diameter and 400 to 600 μm in length

This device may be manufactured by injection molding and in a preferred embodiment, it may be manufactured from polymethylmethacrylate (PMMA).

Further, the probe guide device of this embodiment may have a curvature of about the curvature of an eye, such that it sits appropriately on the given curvature of a patient's eye.

In yet another preferred embodiment, the probe guide device further comprises at least one phalange. Preferably, the phalange has at least one suction cup or suction assembly. More preferably, the device has at least four phalanges and each phalange further has at least two suction cups for immobilizing the device on the patient's cornea. The device may also be immobilized with the aid of at least a partially annular suction ring.

Another embodiment of the present invention provides a biocompatible ophthalmic conductive keratoplasty probe guide device, having:
(1) an arcuate member having a top surface and a bottom surface, wherein the arcuate member has at least one orifice between the top and the bottom surfaces and one cross hair on the top or the bottom surface; and
(2) at least one phalange on the bottom surface of the arcuate member, or
(3) at least a partially annular suction ring around the periphery of the arcuate member.

As before, in this embodiment too, the orifice is substantially cylindrical in shape and preferably the phalange has at least one suction cup or suction assembly to immobilize the device on the patient's cornea. Also, in a preferred embodiment, the at least partially annular suction ring is complete, such that uniform suction may be applied for immobilizing the device on the corneal surface during the procedure.

Another embodiment of the present invention provides a method of guiding a opthalmic probe through a probe guide device on a patient's cornea. As described before, in this method, the opthalmic probe guide device comprises an arcuate member with a top surface and a bottom surface, wherein the arcuate member has at least one orifice between the top and the bottom surfaces and at least one alignment index, such as a cross hair, on the top or the bottom surface. The method preferably comprises the steps of:
(1) placing the probe guide device on the center of a patient's pupil by aligning the cross hair and center of the pupil;
(2) inserting the CK probe through the orifice of the probe guide device, at about 90° angle of incidence; and
(3) applying radiofrequency (RF) energy on the patient's cornea through the CK probe, whereby desirable refractive changes are obtained on the surface of the cornea.

Further, when the probe guide device includes at least one phalange on the bottom surface of the arcuate member having at least one suction cup, then the method of guiding the probe also comprises the step of gently applying pressure on the phalange, after step (1), such that the suction cup immobilizes the device on the patient's cornea.

When the probe guide device includes at least a partially annular suction ring around the periphery of the arcuate member, then the method of guiding the probe also comprises the step of gently applying pressure on the at least partial annular suction ring, after step (1), such that the suction ring immobilizes the device on the patient's cornea. The device may also be immobilized using a suction assembly having a catheter.

Other objects and advantages of the present invention will be apparent from the specification and appended drawing and claims associated with the present invention.

### BRIEF DESCRIPTION OF DRAWINGS

FIG. 1 is a schematic diagram of the distal end 100 of a exemplary commercially available CK probe, showing a tip 110 having a length a and diameter D1, a base 120 having a length b and a diameter D2, a shaft 140 having a diameter D3 and a tapered portion 130 that connects the base 120 and the shaft 140. Typically, the diameter D1 of the tip 110 ranges from 80-120 μm, preferably from 90-110 μm, Typically, the length a of the tip 110 ranges from 400-600 μm, preferably from 450-550 μm, Typically, the diameter D2 of the base 120 ranges from 400-600 μm, preferably from 450-550 μm, Typically, the length b of the base 120 ranges from 400-600 μm, preferably from 450-550 μm,

FIG. 2 depicts one preferred embodiment of the present invention: (A) is a top view of the probe guide device; (B) bottom view of the probe guide device.

FIG. 3 depicts various views of the probe guide device of FIG. 2: (A) partially elevated top perspective view of the proximal end of the probe guide device; (B) top view of the probe guide device; (C) yet another partially elevated top perspective view of the proximal end of the probe guide device; (D); partially elevated bottom perspective view of the proximal end of the probe guide device; (E) bottom view of the probe guide device; (F) yet another partially elevated bottom perspective view of the proximal end of the probe guide device.

FIG. 4 depicts another preferred embodiment of the present invention having at least one suction cup: (A) is a top view of the probe guide device, showing at least two suction cups at 0, 90, 180 and 270 degrees; (B) bottom view of the probe guide device, also showing at least two suction cups at 0, 90, 180 and 270 degrees.

FIG. 5 depicts various views of the probe guide device of FIG. 4: (A) top view of the probe guide device; (B) a partially elevated top perspective view of the proximal end of the probe guide device; (C) bottom view of the probe guide device; (E) partially elevated bottom perspective view of the proximal end of the probe guide device.

FIG. 6 depicts a cross section of a suction assembly presented in one embodiment of the invention for use with the ophthalmic probe guide.

### DETAILED DESCRIPTION OF PREFERRED EMBODIMENTS

A: General

Before the present methods are described, it is understood that this invention is not limited to the particular methodologies, protocols, techniques, and preferred embodiments of the invention as described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “an orifice” includes a plurality of such orifices and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably. As used herein, radiofrequency thermal keratoplasty (RFTK) includes conductive keratoplasty.

Unless defined otherwise, all technical and scientific terms used herein have the same meanings as commonly understood by one of ordinary skill in the art to which this invention belongs. Although any devices, methods and materials similar or equivalent to those described herein can be used in the practice or testing of the present invention, the preferred devices, methods and materials are now described. All publications mentioned herein are incorporated herein by reference for the purpose of describing and disclosing the manufacturing techniques, materials, instruments, and methodologies which are reported in the publications which might be used in connection with the invention. Nothing herein is to be construed as an admission that the invention is not entitled to antedate such disclosure by virtue of prior invention.

B. The Invention

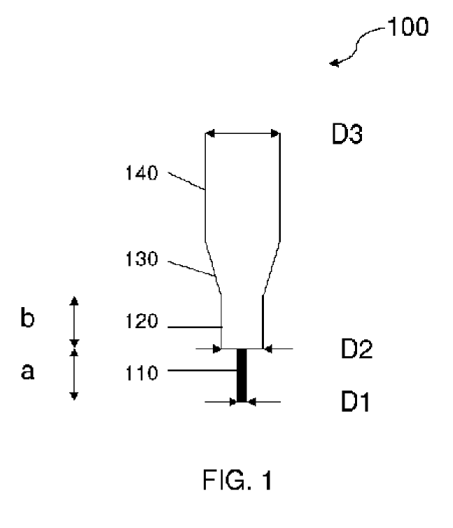

The present invention provides a probe guide device for enhancing the accuracy and placement of an ophthalmic surgical instrument, such as a CK probe during surgery. FIG. 1 is a schematic diagram of the distal end 100 of a exemplary commercially available CK probe, showing a tip 110 having a length a and diameter D1, a base 120 having a length b and a diameter D2, a shaft 140 having a diameter D3 and a tapered portion 130 that connects the base 120 and the shaft 140. Typically, the diameter D1 of the tip 110 is in the range of about 80 to about 120 μm, preferably from about 90 to about 110 μm, Typically, the length a of the tip 110 ranges from about 400 to about 600 μm, preferably from about 450 to about 550 μm. Typically, the diameter D2 of the base 120 ranges from about 400 to about 600 μm, preferably from about 450 to about 550 μm. Typically, the length b of the base 120 ranges from about 400 to about 600 μm, preferably from about 450 to about 550 μm,

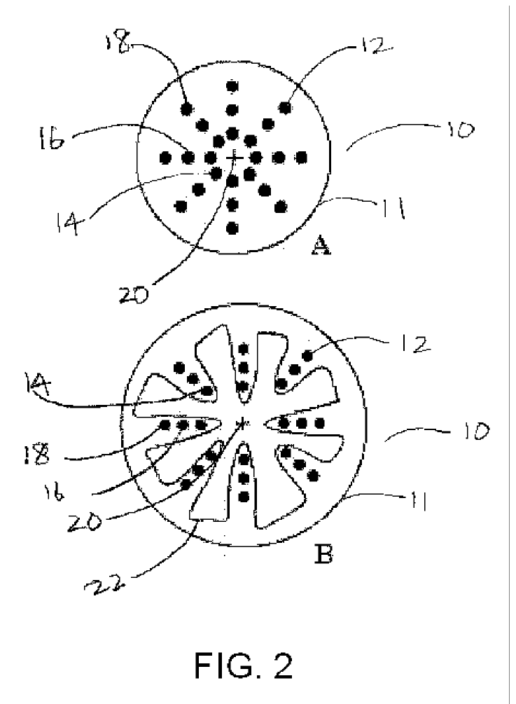

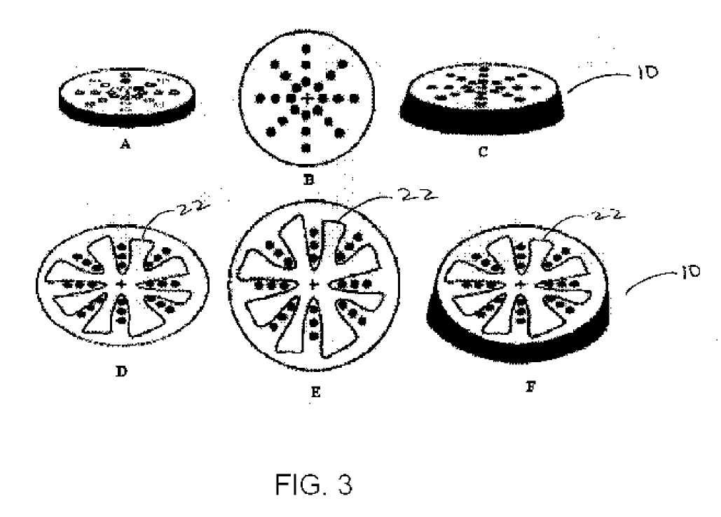

One preferred embodiment of the present invention, as depicted by FIG. 2 and FIG. 3, provides a CK probe guide device 10, which incorporates a transparent, partially arched circular shaped substantially arcuate member 11 with a plurality of orifices 12. Preferably, the arcuate member 11 has a top surface and a bottom surface and eight openings at 6, 7, and 8 mm radius (14,16, and 18 respectively) and an alignment index such as a cross hair 20 at the center to assist with pupil alignment. Each of these orifices 12 defined by a substantially cylindrical sidewall extending through the thickness of the arcuate member from the top surface and a bottom surface is designed to be substantially perpendicular to the cornea and preferably in a symmetrical orientation to each other. The orifices 12 may have specific depth, thus creating a consistent application depth for the CK RF energy.

As seen in FIG. 2B and FIGS. 3D, 3E and 3F, in one preferred embodiment, the device 10 is contoured along the contour lines 22, so as to have a curvature to fit the curvature of an eye upon which the surgery is to be performed. Accordingly, the device 10 may be manufactured with various contours to fit various eye curvatures that may be encountered during a CK surgery. There are numerous patents and publications, such as U.S. Pat. Nos. 4,564,484, 4,787,732 and 6,733,125 that discuss how to a manufacture a lens of a given curvature and what is an appropriate curvature that is suitable for a device that sits on the cornea, such as contact lenses, which are incorporated herein by reference in their entirety for all purposes.

Further, in another preferred embodiment the present guide device 10 may not have distinct contour lines 22, however the curvature may be achieved by molding the device 10 in a similar fashion as a contact lens.

The orifice 12 is preferably designed to be substantially perpendicular to the cornea, such that when a probe is placed through the orifice 12, the angle of incidence is about 90°. This orifice design eliminates surgical error caused by tilting the CK probe during surgery. Further, the orifice 12 is designed to have a certain depth and aperture, which may be based on various factors. The depth of the orifice 12 is defined by the distance between the top and the bottom surfaces of the arcuate member 11. Each orifice 12 is substantially cylindrical in shape, having a inner diameter slightly greater than the outer diameter (D1 in FIG. 1) of the CK probe tip. In certain embodiments, the orifice depth may be slightly less than the length (a in FIG. 1) of the CK probe tip. For example if the Keratoplast™ Tip, which has typical dimensions of about 90μm in diameter and 450 μm in length, is used for delivering the RF energy, then the orifice 12 can have a depth of about 400-500 μm and diameter of about 95-105 μm. This orifice 12 depth and width allows for light touch CK in which the tip of the probe is placed into the cornea. Typically the standard CK method would require the orifice 12 dimensions to be 400 to 600 μm in diameter and 400 to 600 μm in length. Accordingly, in another preferred embodiment, width and depth of the orifice 12 includes a diameter of 400 to 600 microns and a depth of 400 to 600 microns. These dimensions are required to accommodate the base of probe, such that standard and more common CK techniques may be performed, where the base of the probe is compressed against the cornea.

In certain preferred embodiments, the tip of the CK probe is inserted into an orifice of the CK probe guide device so that the distal tip 110 of the probe indents the cornea to the extent that the base 120 of the probe tip is flush with the corneal surface. The RF energy is applied while the base of the probe tip is flush with and compressing the cornea. In such embodiments, the orifice of the CK probe guide device has an internal diameter suitable to accommodate the outside diameter of the base of the probe tip, typically about 400-600 μm, and preferably about 450-550 μm and a depth about the length to the base of the probe tip, typically about 400-600 μm, and preferably about 450-550 μm.

In other embodiments, the depth of the orifice is less than the length of the tip of the CK probe, (e.g., Keratoplast™ Tip), such that the distal tip of the CK probe is completely inserted through the orifice, as desired in certain RF applications. In such embodiments, the orifice of the CK probe guide device has an internal diameter suitable to accommodate the outside diameter of the probe tip, typically 80-120 μm, preferably about 90-110μm, more preferably about 95-105 μm and a depth that can be less than the distance to the junction of the tip and the base of the probe tip, typically less than about 400-600 μm, and preferably less than about 400-500 μm.

In practice, a surgeon may measure the corneal curvature of a given patient and select a suitably contoured guide 10. This device 10 then may be centered and aligned on the eye by placing the alignment index, such as a cross hair 20 on to the center of a patient's pupil. CK may be performed on select points by placing the CK probe through a select orifice 12. The orifice 12 may be selected based on where the refractive changes in the cornea are required.

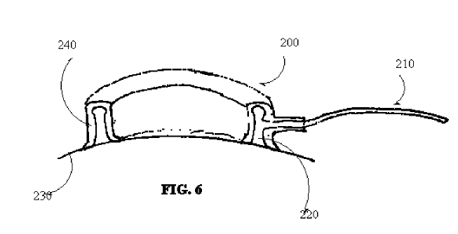

Suction devices may be used to keep the arcuate member 11 in position. Such devices include a phalange 26 at the bottom of the arcuate member 11 having a suction cup 24, or an annular suction ring 28 that may be used along the periphery of the device 10 or a suction assembly 200 that also may be used along the periphery of the device 10, see FIG. 6.

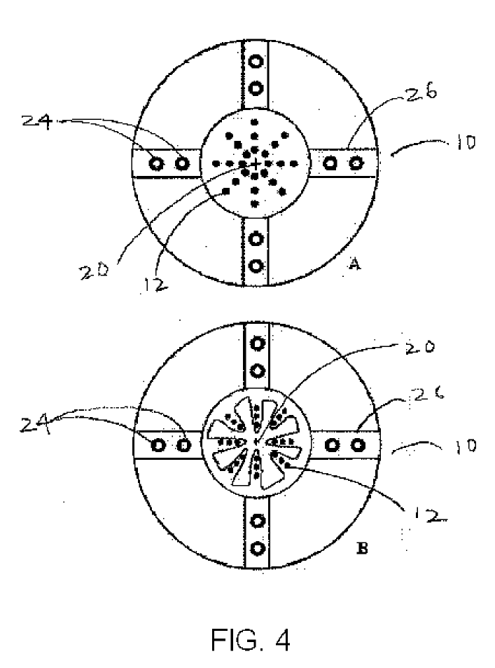

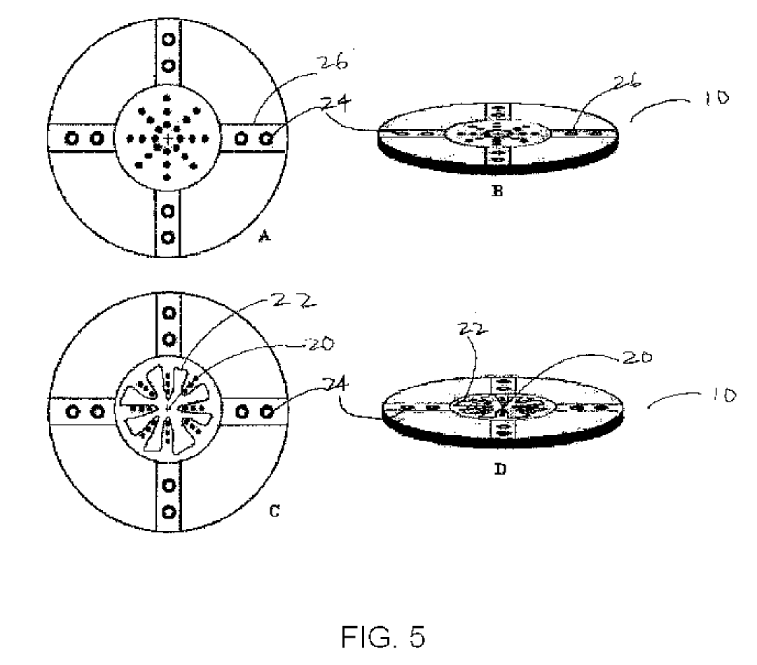

Further, in one embodiment, as shown in FIGS. 4 and 5, the bottom of the arcuate member 11 may have at least one phalange 26, with at least one suction device, such as a suction cup 24 to keep the device 10 stationary and immobile during the surgery. The suction devices such as the annular suction ring 28 or the suction assembly 200 may have a catheter attached to the device by which suction is applied. In one embodiment, the suction device may be similar to that found in the lasik microkeratome suction ring. A suction device, such as a suction ring 28 is well known in the art, and is commercially available, for example, INTRALASE® FS (Intralase Corp., Irvine Calif.). Other suction devices such as suction assembly 200 may also be used in place of a suction ring 28 in order to keep the device 10 stationary and immobile during surgery. Preferably, as shown in FIG. 6, the suction assembly 200 has an annular suction ring 240 connected to a catheter 210. Further, this suction assembly 200 has a lumen 220, preferably a substantially circular lumen in communication with the corneal surface 230 such that it provides a passage for creating suction via the catheter 210.

The suction device such as the suction cup 24 is positioned at the bottom of the phalange 26. The arcuate member 11 may have at least one pair of phalanges 26 located diametrically opposite to each other. Most preferably these phalanges 26 are radially arranged, as shown in FIGS. 4B and 5C and 5D, however, these phalanges 26 may be arranged in any orientation such that the suction cup 24 may hold the device 10 in an immobile, stationary position relative to the conjunctiva, without altering the curvature of the cornea. In another embodiment, the bottom of the arcuate member 11 includes at least four phalanges 26, and each phalange 26, further includes a pair of suction cups 24.

The suction cups 24 are also symmetrically positioned along the phalange 26, such that each suction cup 24 is capable of applying uniform suction to the conjunctiva, without altering the curvature of the cornea. The suction cup 24 is analogous to the underside of an octopus' suction cup.

In another embodiment, the device 10 may be held in position and immobilized by an annular suction ring 28 (not shown) along the periphery of the arcuate member 11.

In practice, this device 10 may be used intraoperatively by first centering the device 10 on to a patient's pupil, as described above. Once the device 10 is centered, mild pressure may be applied to the suction cup 24 or the suction ring 28, or suction may be applied through the catheter 210 of the suction assembly 200 to immobilize the device 10, thus negating the effects of patient movement and surgical application. For performing CK, the CK probe, as described above may be positioned on select orifice 12 before applying desirable amount RF energy.

Materials and techniques suitable for manufacturing this device 10 include all polymeric materials and molding techniques known to one of ordinary skill in the art useful for manufacturing CK markers and contact lenses (e.g., intralase suction ring or suction rings used with keratomes in LASIK procedures). Such polymers and techniques include injection molded plastic such as polymethylmethacrylate (PMMA), hydroxyethylmethacrylate (HEMA), silicone polymers, fluorocarbon copolymers or vinyl pyrrolidone.

Generally, the present invention generally provides a biocompatible conductive keratoplasty probe guide device 10 having an arcuate member 11 and at least one orifice 12 capable of allowing the probe to be inserted through the orifice 12. Also, the present invention teaches methods related to guiding a CK probe through this probe guide device 10.

In one preferred embodiment, the biocompatible ophthalmic Conductive Keratoplasty (CK) probe guide device 10 comprises an arcuate member 11 having a top surface and a bottom surface. In this device 10 the arcuate member 11 has at least one orifice 12 between the top and the bottom surfaces and one cross hair 20 on the top or the bottom surface.

Preferably, the device 10 has at least twenty-four orifices 12 arranged in a symmetrical pattern. The arrangement of the orifices 12 may be as follows: the first eight orifices 12 are located at about 6 mm distance from the center of the cross hair 20, the second eight orifices 12 are located at about 7 mm distance from the center of the cross hair 20 and the last eight orifices 12 are located at about 8 mm distance from the center of the cross hair 20. This arrangement forms eight radial arrays of three orifices 12 each. Further, the eight radial arrays are preferably substantially equiangularly positioned at 45° to each other.

In another embodiment, the probe guide device 10 has at least sixteen orifices about 6.5 mm distance from the center of the cross hair 20 and the second eight orifices 12 are located at about 7.5 mm distance from the center of the cross hair 20, thus forming eight radial arrays of two orifices 12 each. Such a device 10 with orifices 12 placed at different distances from the cross hair 20 may be used after a person has had CK and needs further enhancement surgery to attain the most desired outcome. If this is the case, the surgeon would place additional RF spots in the cornea but at a different placement point. As one of ordinary skill in the art would realize, the placement of orifices may be altered to either suit the size of the cornea or to place additional RF spots. While the invention has been described to have orifice 20 placements at 6, 6.5, 7, 7.5 or 8 mm from the cross hair 20, these placements may be easily altered without undue experimentation and routine procedures.

Also, in certain embodiments of this device 10, the orifice 12 is substantially cylindrical in shape. Preferably, the orifice 12 has a diameter of in the range of about 90 to about 100 μm and length in the range of about 450 to about 500 μm.

This device 10 may be manufactured by injection molding and in a preferred embodiment, it may be manufactured from PMMA, polymethylmethacrylate.

Further, the probe guide device 10 of this embodiment may have a curvature of about the curvature of an eye, such that it sits appropriately on the given curvature of a patient's eye.

In yet another preferred embodiment, the probe guide device 10 further comprises at least one phalange 26. Preferably, the phalange 26 has at least one suction cup 24. More preferably, the device 10 has at least four phalanges 26 and each phalange 26 further has at least two suction cups 24 for immobilizing the device 10 on the patient's cornea. The device 10 may also be immobilized with the aid of at least a partially annular suction ring 28 or a suction assembly 200.

Another embodiment of the present invention provides a biocompatible ophthalmic Conductive Keratoplasty probe guide device 10, having:
(1) an arcuate member 11 having a top surface and a bottom surface, wherein the arcuate member 11 has at least one orifice 12 between the top and the bottom surfaces and one alignment index, such as a cross hair 20, on the top or the bottom surface; and
(2) at least one phalange 26 on the bottom surface of the arcuate member 11, or
(3) at least a partially annular suction ring 28 around the periphery of the arcuate member 11; or
(4) a suction asembly.

As before, in this embodiment too, the orifice 12 is substantially cylindrical in shape and preferably the phalange 26 has at least one suction cup 24 to immobilize the device 10 on the patient's cornea. Also, in a preferred embodiment, the at least partially annular suction ring 28 is complete, such that uniform suction may be applied for immobilizing the device 10.

Another embodiment of the present invention provides a method of guiding a CK probe through a probe guide device 10 on a patient's cornea. As described before, in this method, the probe guide device 10 comprises an arcuate member 11 with a top surface and a bottom surface, wherein the arcuate member 11 has at least one orifice 12 between the top and the bottom surfaces and one alignment index, such as a cross hair 20, on the top or the bottom surface. The method preferably comprises the steps of:
(1) placing the probe guide device 10 on the center of a patient's pupil by aligning the cross hair 20 and center of the pupil;
(2) inserting the CK probe through the orifice 12 of the probe guide device 10, at about 90° angle of incidence; and
(3) applying radiofrequency (RF) energy on the patient's cornea through the CK probe, whereby desirable refractive changes are obtained on the surface of the cornea.

Further, when the probe guide device 10 includes at least one phalange 26 on the bottom surface of the arcuate member 11 having at least one suction cup 24, then the method of guiding the probe also comprises the step of gently applying pressure on the phalange 26, after step (1), such that the suction cup 24 immobilizes the device 10 on the patient's cornea.

When the probe guide device 10 includes at least a partially annular suction ring 28 around the periphery of the arcuate member 11, then the method of guiding the probe also comprises the step of gently applying pressure on the at least partial annular suction ring 28, after step (1), such that the suction ring immobilizes the device 10 on the patient's cornea.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A biocompatible ophthalmic surgical probe guide device, comprising:
(a) an arcuate member having a top surface and a bottom surface, wherein the arcuate member has at least one orifice between the top and the bottom surfaces disposed to admit and align a tip of an ophthalmic surgical probe and
(b) one alignment index on the top or the bottom surface.

2. The probe guide device of claim 1, wherein the ophthalmic surgical probe is a radiofrequency thermal keratoplasty probe.

3. The probe guide device of claim 1, wherein the device has at least 24 orifices arranged in a symmetrical pattern whereby the first 8 orifices are located at about 6 mm distance from the center of the cross hair, the second 8 orifices are located at about 7 mm distance from the center of the cross hair and the last 8 orifices are located at about 8 mm distance from the center of the cross hair, thereby forming 8 radial arrays of 3 orifices each.

4. The probe guide device of claim 3, wherein the 8 radial arrays are substantially equiangularly positioned at 45° to each other.

5. The probe guide device of claim 1, wherein the device has at least 16 orifices arranged in a symmetrical pattern whereby the first 8 orifices are located at about 6.5 mm distance from the center of the cross hair and the second 8 orifices are located at about 7.5 mm distance from the center of the cross hair, thereby forming 8 radial arrays of 2 orifices each.

6. The probe guide device of claim 5, wherein the 8 radial arrays are substantially equiangularly positioned at 45° to each other.

7. The probe guide device of claim 1, wherein the orifice is substantially cylindrical in shape and has a diameter of about 80 to about 600 μm and depth of about 400 to about 600 μm.

8. The probe guide device of claim 1, wherein the device has a curvature of about the curvature of a cornea.

9. The probe guide device of claim 1, wherein the device further comprises at least one phalange.

10. The probe guide device of claim 9, wherein the phalange has at least one suction cup.

11. The probe guide device of claim 10, wherein the device has at least four phalanges and each phalange further has at least two suction cups.

12. The probe guide device of claim 1, wherein the device further comprises a suction assembly.

13. The probe guide device of claim 1, wherein the device further comprises at least one partially annular suction ring.

14. A biocompatible ophthalmic surgical probe guide device, comprising:
(a) an arcuate member having a top surface and a bottom surface, wherein the arcuate member has at least one orifice between the top and the bottom surfaces and at least one alignment index on the top or the bottom surface; and
(b) at least one phalange on the bottom surface of the arcuate member, or
(c) at least a partially annular suction ring around the periphery of the arcuate member
(d) a suction assembly around the periphery of the arcuate member.
